(19) United States
(12) Patent Application Publication
(10) Pub. No.: US 2024/0173418 A1
(43) Pub. Date: May 30, 2024

(54) CYCLIC DINUCLEOTIDE CONJUGATES AND RELATED METHODS OF USE THEREOF

(71) Applicant: The Regents of the University of Michigan, Ann Arbor, MI (US)

(72) Inventors: James J. Moon, Ann Arbor, MI (US); Xiaoqi Sun, Ann Arbor, MI (US)

(21) Appl. No.: 18/284,035

(22) PCT Filed: Mar. 23, 2022
(86) PCT No.: PCT/US2022/021524

§ 371 Date: Sep. 25, 2023

Related U.S. Application Data
(60) Provisional application No. 63/165,969, filed on Mar. 25, 2021.

(51) Int. Cl. A61K 47/54; A61K 45/06; A61K 49/00; A61K 49/08; A61K 49/10; A61K 51/04

(52) U.S. Cl. CPC A61K 47/549; A61K 45/06; A61K 49/0032; A61K 49/0052; A61K 49/085; A61K 49/10; A61K 51/0491

(57) ABSTRACT

This invention is in the field of medicinal chemistry and relates to a new class of cyclic dinucleotide conjugates (e.g., Formula I) which function as tumor-targeted imaging agents, and their use in diagnostic and therapeutic intervention for disorders (e.g., cancer).

### CROSS REFERENCE TO RELATED APPLICATIONS

This application claims benefit of priority to U.S. Provisional Application No. 63/165,969, filed Mar. 25, 2021, the contents of which are incorporated herein by reference in their entirety.

### FIELD OF THE INVENTION

This invention is in the field of medicinal chemistry and relates to a new class of cyclic dinucleotide conjugates (e.g.,

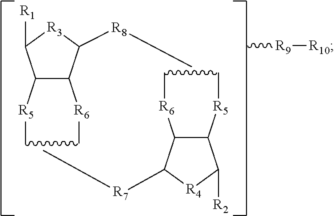

Formula I) which function as tumor-targeted imaging agents, and their use in diagnostic and therapeutic intervention for disorders (e.g., cancer).

### INTRODUCTION

Tumor-targeted imaging is an emerging strategy for in vivo visualization of cancer over time based on biological differences between cancer normal tissues. Available imaging tools include PET imaging, CT imaging, MR imaging, ultrasonic imaging, and fluorescence imaging. They are powerful tools for accurate diagnosis, staging, and restaging of cancer. For example, F18-FDG has been widely used for diagnosing cancer and tracking cancer development during therapy. However, most of the current imaging agents are distributed throughout the body, resulting in accumulation in normal healthy tissues. This results in low signal to noise ratio and causes side effects.

There are intensive efforts to develop intra-operational imaging that could aid surgeons in identifying and distinguishing tumors from healthy tissues. For example, a folate-FITC conjugate has been developed to visualize tumors with high folic acid receptor-α, (FAR-α) expression. To develop imaging agents that could realize efficient intra-operational imaging, highly selective ligands of cancer cells should be developed to achieve high tumor/normal tissue contrast as well (see, van Dam., G.M., et al., Nat Med 17, 1315-1319, 2011).

Pharmaceutical agents that are conjugated to tumor-targeted ligands could also be delivered to tumor tissue specifically like a “magic bullet”. For example, several antibody-drug conjugates have been approved for treatment of cancers and have been proved to be very efficient.

Thus, developing tumor-targeting agents with a high selectivity to cancer tissues over normal tissues would greatly improve the clinical diagnosis and therapeutic intervention of cancer.

The present invention addresses these needs.

### SUMMARY

Experiments conducted during the course of developing embodiments for the present invention arrived at a surprising discovery that cyclic dinucleotides (CDNs) conjugated with imaging and/or diagnostic moieties selectively accumulate in tumor tissues after systemic administration with a high signal to noise ratio. Several CDNs (including cGAMP and CDG) were used to show that they accumulate in tumors with high efficiency. In addition, CDNs conjugated with different signaling moieties (including Cy7 and Dy547) were shown to accumulate in tumors with high efficiency.

Accordingly, the present invention relates to a new class of cyclic dinucleotide conjugates which function as tumor-targeted imaging agents, and their use in diagnostic and therapeutic intervention for disorders (e.g., cancer).

Certain cyclic dinucleotide conjugates of the present invention may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

In a particular embodiment, compounds encompassed within the following formula is provided:

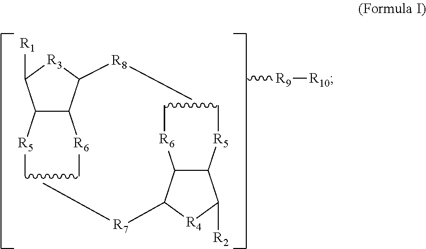

including pharmaceutically acceptable salts, solvates, and/or prodrugs thereof.

Certain cyclic dinucleotide conjugates of the present invention may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

Formula I is not limited to a particular chemical moiety for R1, R2, R3, R4, R5, R6, R7, R8, R9 and R10. In some embodiments, the particular chemical moiety for R1, R2, R3, R4, R5, R6, R7, R8, R9 and R10 independently include any chemical moiety that permits the resulting cyclic dinucleotide conjugate to effectively function within cancer imaging, cancer diagnosis and cancer-targeted delivery of therapeutics. In some embodiments, the particular chemical moiety for R1, R2, R3, R4, R5, R6, R7, R8, R9 and R10 independently include any chemical moiety that permits the resulting cyclic dinucleotide conjugate to be used as a contrast agent for PET imaging of cancer, wherein the CDN or CDN conjugates were radiolabeled with radioactive isotopes. In some embodiments, the particular chemical moiety for R1, R2, R3, R4, R5, R6, R7, R8, R9 and R10 independently include any chemical moiety that permits the resulting cyclic dinucleotide conjugate to be used as contrast agents of MR imaging of cancer, wherein, the CDN are conjugated with an MR1 moiety (e.g., DOTA-Gd/Mn, NOTA-Gd/Mn, etc). In some embodiments, the particular chemical moiety for R1, R2, R3, R4, R5, R6, R7, R8, R9 and R10 independently include any chemical moiety that permits the resulting cyclic dinucleotide conjugate to be used for imaging purposes (e.g., PET imaging, MR imaging, fluorescent imaging, photoacoustic imaging, acoustic imaging, etc). In some embodiments, the particular chemical moiety for R1, R2, R3, R4, R5, R6, R7, R8, R9 and R10 independently include any chemical moiety that permits the resulting cyclic dinucleotide conjugate to be used for diagnostic purposes (e.g., histochemical staining of tumor, intraoperative imaging of tumor to aid surgery therapy, predicting prognosis, etc.). In some embodiments, the particular chemical moiety for R1, R2, R3, R4, R5, R6, R7, R8, R9 and R10 independently include any chemical moiety that permits the resulting cyclic dinucleotide conjugate to be used for therapeutic purposes (e.g., wherein the cyclic dinucleotide conjugate is conjugated with a therapeutic agent (e.g., a cancer therapeutic agent)).

Such embodiments are not limited to a particular definition for R1 and R2. In some embodiments, R1 and R2 are each independently a nucleotide moiety. For example, in some embodiments, R1 and R2 are each independently selected from adenine, cytosine, guanine, thymine, inosine, purine, and any derivative thereof.

Such embodiments are not limited to a particular definition for R3 and R4. In some embodiments, R3 and R4 are each independently selected from Sulfur, Nitrogen, Oxygen, and CH₂.

Such embodiments are not limited to a particular definition for R5 and R6. In some embodiments, R5 and R6 are each independently selected from NH, CH₂, Oxygen, Fluorine, Iodine, an isotope (e.g., radioisotope (eg., ₁₈F, ₁₂₃I, ₁₂₄I, ₁₂₅I, ₃₂P, ₃₅S, ₆₇Ga, etc)), a chromogenic enzyme (e.g., peroxidase, alkaline phosphatase, etc), a chromophore, a luminescent or fluorescent substance (e.g., FITC, RITC, green fluorescent protein (GFP), enhanced green fluorescent protein (EGFP), red fluorescent protein (RFP), discosoma sp. red fluorescent protein (DsRed), cyan fluorescent protein (CFP), cyan green fluorescent protein (CGFP), yellow fluorescent protein (YFP), Cy3, Cy5, Cy7.5, etc), and a magnetic resonance imaging substance (e.g., gadolinium (Gd), super paramagentic particles or ultrasuper paramagentic particles, etc).

Such embodiments are not limited to a particular definition for R7 and R8. In some embodiments, R7 and R8 are each independently selected from

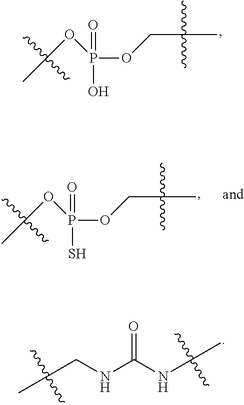 [SMILES: *COP(=O)(O)O* ; *COP(=O)(S)O* ; *CNC(=O)N*]

Such embodiments are not limited to a particular definition for R9. In some embodiments, R9 is a peptide linker or a non-peptide linker that covalently links the R10 to the one of R1, R2, R5 and R6. In some embodiments, R9 is selected from

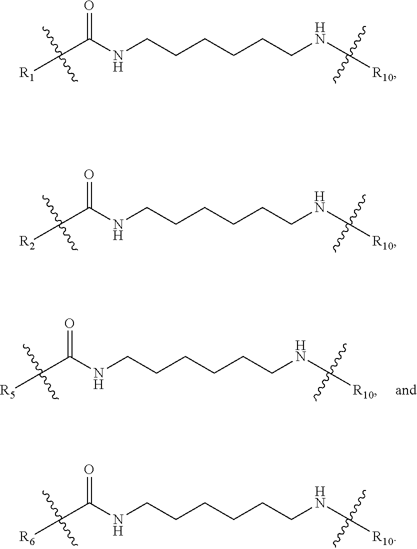

Such embodiments are not limited to a particular definition for R10. In some embodiments, R10 is selected from a therapeutic agent, a biological monitoring agent, an imaging agent, and a targeting agent. In some embodiments, R10 is Cy7. In some embodiments, R10 is Dy547.

In some embodiments, the therapeutic agent is selected from a chemotherapeutic agent, an anti-oncogenic agent, an anti-angiogenic agent, a tumor suppressor agent, an anti-microbial agent, or an expression construct comprising a nucleic acid encoding a therapeutic protein, although the present invention is not limited by the nature of the therapeutic agent. In further embodiments, the therapeutic agent is protected with a protecting group selected from photo-labile, radio-labile, and enzyme-labile protecting groups. In some embodiments, the chemotherapeutic agent is selected from a group consisting of, but not limited to, platinum complex, verapamil, podophylltoxin, carboplatin, procarbazine, mechloroethamine, cyclophosphamide, camptothecin, ifosfamide, melphalan, chlorambucil, bisulfan, nitrosurea, adriamycin, dactinomycin, daunorubicin, doxorubicin, bleomycin, plicomycin, mitomycin, bleomycin, etoposide, tamoxifen, paclitaxel, taxol, transplatinum, 5-fluorouracil, vincristin, vinblastin, bisphosphonate (e.g., CB3717), chemotherapeutic agents with high affinity for folic acid receptors, ALIMTA (Eli Lilly), and methotrexate. In some embodiments, the anti-oncogenic agent comprises an antisense nucleic acid (e.g., RNA, molecule). In certain embodiments, the antisense nucleic acid comprises a sequence complementary to an RNA of an oncogene. In preferred embodiments, the oncogene includes, but is not limited to, abl, Bcl-2, Bcl-xL, erb, fms, gsp, hst, jun, myc, neu, raf; ras, ret, src, or trk. In some embodiments, the nucleic acid encoding a therapeutic protein encodes a factor including, but not limited to, a tumor suppressor, cytokine, receptor, inducer of apoptosis, or differentiating agent. In preferred embodiments, the tumor suppressor includes, but is not limited to, BRCA1, BRCA2, C-CAM, p16, p21, p53, p73, Rb, and p27. In preferred embodiments, the cytokine includes, but is not limited to, GMCSF, IL-1, IL-2, IL-3, IL-4, IL-5, IL-6, IL-7, IL-8, IL-9, IL-10, IL-11, IL-12, IL-13, IL-14, IL-15, β-interferon, γ-interferon, and TNF. In preferred embodiments, the receptor includes, but is not limited to, CFTR, EGFR, estrogen receptor, IL-2 receptor, and VEGFR. In preferred embodiments, the inducer of apoptosis includes, but is not limited to, AdElB, Bad, Bak, Bax, Bid, Bik, Bim, Harakid, and ICE-CED3 protease. In some embodiments, the therapeutic agent comprises a short-half life radioisotope.

In some embodiments, the biological monitoring agent comprises an agent that measures an effect of a therapeutic agent (e.g., directly or indirectly measures a cellular factor or reaction induced by a therapeutic agent), however, the present invention is not limited by the nature of the biological monitoring agent. In some embodiments, the monitoring agent is capable of detecting (e.g., measuring) apoptosis caused by the therapeutic agent.

In some embodiments, the imaging agent comprises a radioactive label including, but not limited to ¹⁴C, 36Cl, ⁵⁷CO, ⁵⁸CO, ⁵¹Cr, ¹²⁵I, ¹³¹I, ¹¹¹Ln, ¹⁵²Eu, ⁵⁹Fe, ⁶⁷Ga, ³²P, ¹⁸⁶Re, ³⁵S, ⁷⁵Se, Tc-99m, and ¹⁷⁵ Yb. In some embodiments, the imaging agent comprises a fluorescing entity. In a preferred embodiment, the imaging agent is fluorescein isothiocyanate or 6-TAMARA. In some embodiments, the imaging agent is applicable for photoacoustic imaging and/or acoustic imaging.

In some embodiments, the targeting agent includes, but is not limited to an antibody, receptor ligand, hormone, vitamin, and antigen, however, the present invention is not limited by the nature of the targeting agent. In some embodiments, the antibody is specific for a disease-specific antigen. In some preferred embodiments, the disease-specific antigen comprises a tumor-specific antigen. In some embodiments, the receptor ligand includes, but is not limited to, a ligand for CFTR, EGFR, estrogen receptor, FGR2, folate receptor, IL-2 receptor, glycoprotein, and VEGFR. In a preferred embodiment, the receptor ligand is folic acid.

In some embodimetns, R1, R2, R3, R4, R5, R6, R7, and R8 independently combine such that the resulting chemical moiety is a cyclic dinucleotide. In some embodiments, the cyclic dinucleotide is selected from cGAMP, cdiAMP, cdiGMP, and cAIMP. In some embodimetns, the cyclic dinucleotide is selected from 2′3′-cGAMP, 3′3′-cGAMP, c-di-AMP, c-di-GMP, cAIMP, cAIMP Difluor, cAIM(PS)2, Difluor (Rp/Sp), 2′2′-cGAMP, 2′3′-cGAM(PS)2 (Rp/Sp), 3′3′-cGAMP Fluorinated, c-di-AMP Fluorinated, 2′3′-c-di-AMP, 2′3′-c-di-AM(PS)2 (Rp,Rp), c-di-GMP Fluorinated, 2′3′-c-di-GMP, c-di-IMP, cGAMP, 2′3′-cGAMP, 2′2′-cGAMP, 3′3′-cGAMP, cGAM(PS)2,2′3′-cGAM(PS)2(Rp/Sp), 2′2′-cGAM(PS)2,2′3′-cGAM(PS)2, cGAMP Fluorinated, 3′3′-cGAMP Fluorinated, 2′3′-cGAMP Fluorinated, 2′2′-cGAMP Fluorinated, c-di-AMP, 2′3′-cdAMP, 2′2′-cdAMP, 3′3′-cdAMP, c-di-AM(PS)2,2′3′-c-di-AM(PS)2 (Rp,Rp), 2′2′-c-di-AM(PS)2, 3′3′-c-di-AM(PS)2, c-di-AMP Fluorinated, 2′3′-cdAMP Fluorinated, 2′2′-cdAMP Fluorinated, 3′3′-cdAMP Fluorinated, cdGMP, 2′3′-cdGMP, 2′2′-cdGMP, 3′3′-cdGMP, c-di-GM(PS)2, 2′3′-c-di-GM(PS)2,2′2′-c-di-GM(PS)2, 3′3′-c-di-GM(PS)2, cdGMP Fluorinated, 2′3′-cdGMP Fluorinated, 2′2′-cdGMP Fluorinated, 3′3′-cdGMP Fluorinated, cAIMP, 2′3′-cAIMP, 2′2′-cAIMP, 3′3′-cAIMP, cAIMP Difluor (3′3′-cAIMP Fluorinated, 2′3′-cAIMP Fluorinated, 2′2′-cAIMP Fluorinated, cAIM(PS)2 Difluor, 3′3′-cAIM(PS)2 Difluor (Rp/Sp), 2′3′-cAIM(PS)2 Difluor, 2′2′-cAIM(PS)2 Difluor, c-di-IMP, 2′3′-cdIMP, 2′2′-cdIMP, 3′3′-cdIMP, c-di-IM(PS)2,2′3′-c-di-IM(PS)2,2′2′-c-di-IM(PS)2, 3′3′-c-di-IM(PS)2, c-di-IMP Fluorinated, 2′3′-cdIMP Fluorinated, 2′2′-cdIMP Fluorinated, 3′3′-cdIMP Fluorinated, etc. Additional examples of cyclic dinucleotides are described in some detail in, e.g., U.S. Pat. Nos. 7,709,458 and 7,592,326; WO2007/054279; and Yan et al., Bioorg. Med. Chem Lett. 18: 5631 (2008)).

In some embodiments, the cyclic dinucleotide conjugate of Formula I is selected from:

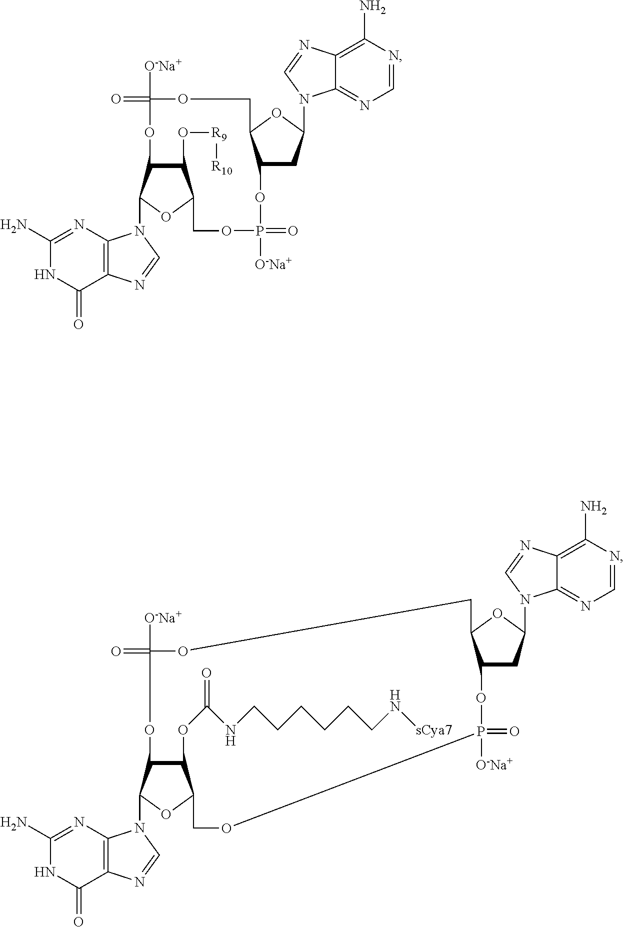

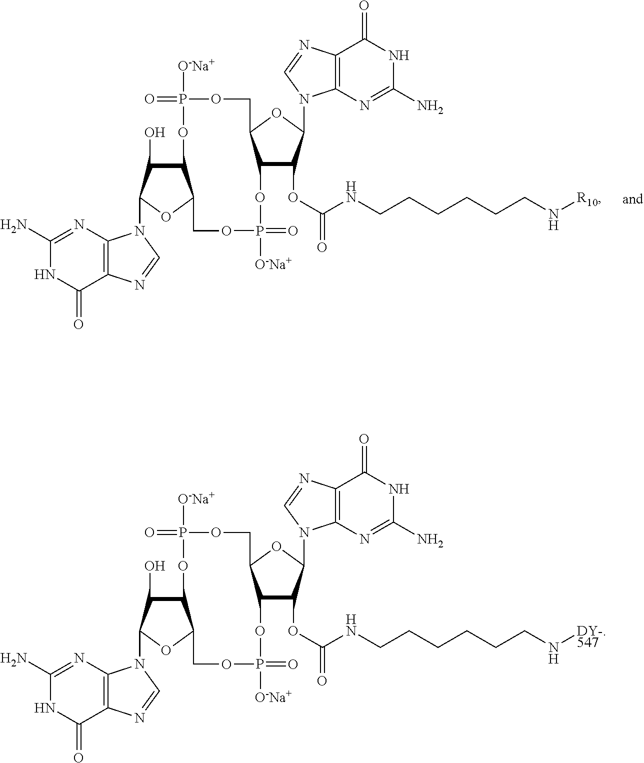

The invention further provides processes for preparing any of the cyclic dinucleotide conjugates of the present invention.

In certain embodiments, the present invention provides methods for administering a pharmaceutical composition comprising one or more cyclic dinucleotide conjugates of the present invention to a subject (e.g., a human subject) (e.g., a human subject suffering from or at risk of suffering from cancer) for purposes of imaging cancer cells (e.g., tumor cells), diagnosing a cancer, and/or treating, preventing and/or ameliorating the symptoms of a cancer.

In certain embodiments, the present invention provides a method of imaging a cancer cell comprising providing a composition comprising cyclic dinucleotides conjugated with an imaging agent as described herein, and exposing the cancer cell to the composition under conditions such that the composition interacts with and renders the cancer cell detectable. In some embodiments, the cancer cell is present in vivo. In some embodiments, the method images the cancer cell in a region beyond the primary tumor site. In some embodiments, detection of the cancer cell in a region beyond the primary tumor site is indicative of metastasis. In some embodiments, the imaging is used for staging of cancer. In some embodiments, the cyclic dinucleotides conjugated with an imaging agent are further conjugated with a therapeutic agent, biological monitoring agent, and/or a targeting agent.

In some embodimetns, the cyclic dinucleotides are conjugated with a radioisotope and are used as a contrast agent in PET imaging applications.

In some embodimetns, the cyclic dinucleotides are conjugated with a MR1 imaging moiety (e.g., DOTA-Gd/Mn, NOTA-Gd/Mn, etc.), and are used as a contrast agent in MRI imaging applications.

The present invention also provides a method of treating a cancer comprising administering to a subject suffering from or susceptible to cancer a therapeutically effective amount of a composition comprising cyclic dinucleotides conjugated with a therapeutic agent. In some embodiments, the cyclic dinucleotides conjugated with a therapeutic agent are further conjugated with an imaging agent, biological monitoring agent, and/or a targeting agent. In some embodiments, the composition is co-administered with an additional therapeutic agent (e.g., chemotherapeutic agent, an anti-oncogenic agent, an anti-angiogenic agent, a tumor suppressor agent, an anti-microbial agent, etc.).

Such methods are not limited by the type of cancer treated using the compositions and methods of the present invention. Indeed, a variety of cancer can be treated including, but not limited to, leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma.

In certain embodiments, the present invention provides kits comprising a pharmaceutical composition comprising a cyclic dinucleotide conjugated as described herein, and one or more of (1) a container, pack, or dispenser, (2) one or more additional agents selected from a chemotherapeutic agent, an anti-oncogenic agent, an anti-angiogenic agent, a tumor suppressor agent, an anti-microbial agent, etc., and (3) instructions for administration.

### BRIEF DESCRIPTION OF THE DRAWINGS

FIG. 1 shows distribution of cGAMP-Cy7 after 24 hr of administration in murine model of CT26 colon carcinoma.

FIG. 2 shows distribution of CDG-Dy547 after 24 hr of administration in murine model of CT26 colon carcinoma.

FIG. 3 shows distribution of cGAMP-Cy7 after 24 hr of administration in murine model of MMTV-PyMT spontaneous breast cancer.

FIG. 4A and 4B shows distribution of cGAMP-Cy7 after 24 hr of administration in murine model of A) NOOC1 subcutaneous head and neck carcinoma, and B) MC38 colon carcinoma.

### DETAILED DESCRIPTION OF THE INVENTION

Experiments conducted during the course of developing embodiments for the present invention arrived at a surprising discovery that cyclic dinucleotides (CDNs) selectively conjugated with imaging and/or diagnostic moieties selectively accumulate in tumor tissues after systemic administration with a high signal to noise ratio. Several CDNs (including cGAMP and CDG) were used to show that they accumulate in tumors with high efficiency. In addition, CDNs conjugated with different signaling moieties (including Cy7 and Dy547) were shown to accumulate in tumors with high efficiency. Moreover, such CDNs selectively conjugated with imaging and/or diagnostic moieties were shown to rapidly clear from the system through kidneys.

Accordingly, the present invention relates to a new class of cyclic dinucleotide conjugates which function as tumor-targeted imaging agents, and their use in diagnostic and therapeutic intervention for disorders (e.g., cancer).

In a particular embodiment, compounds encompassed within the following formula is provided:

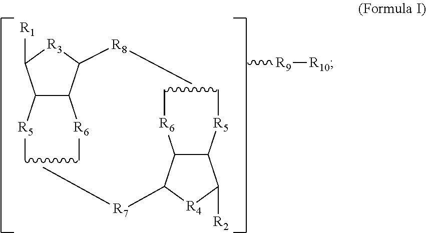

including pharmaceutically acceptable salts, solvates, and/or prodrugs thereof.

Certain cyclic dinucleotide conjugates of the present invention may exist as stereoisomers including optical isomers. The invention includes all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

Formula I is not limited to a particular chemical moiety for R1, R2, R3, R4, R5, R6, R7, R8, R9 and R10. In some embodiments, the particular chemical moiety for R1, R2, R3, R4, R5, R6, R7, R8, R9 and R10 independently include any chemical moiety that permits the resulting cyclic dinucleotide conjugate to effectively function within cancer imaging, cancer diagnosis and cancer-targeted delivery of therapeutics. In some embodiments, the particular chemical moiety for R1, R2, R3, R4, R5, R6, R7, R8, R9 and R10 independently include any chemical moiety that permits the resulting cyclic dinucleotide conjugate to be used as a contrast agent for PET imaging of cancer, wherein the CDN or CDN conjugates were radiolabeled with radioactive isotopes. In some embodiments, the particular chemical moiety for R1, R2, R3, R4, R5, R6, R7, R8, R9 and R10 independently include any chemical moiety that permits the resulting cyclic dinucleotide conjugate to be used as contrast agents of MR imaging of cancer, wherein, the CDN are conjugated with an MR1 moiety (e.g., DOTA-Gd/Mn, NOTA-Gd/Mn, etc). In some embodiments, the particular chemical moiety for R1, R2, R3, R4, R5, R6, R7, R8, R9 and R10 independently include any chemical moiety that permits the resulting cyclic dinucleotide conjugate to be used for imaging purposes (e.g., PET imaging, MR imaging, fluorescent imaging, photoacoustic imaging, acoustic imaging, etc). In some embodiments, the particular chemical moiety for R1, R2, R3, R4, R5, R6, R7, R8, R9 and R10 independently include any chemical moiety that permits the resulting cyclic dinucleotide conjugate to be used for diagnostic purposes (e.g., histochemical staining of tumor, intraoperative imaging of tumor to aid surgery therapy, predicting prognosis, etc.). In some embodiments, the particular chemical moiety for R1, R2, R3, R4, R5, R6, R7, R8, R9 and R10 independently include any chemical moiety that permits the resulting cyclic dinucleotide conjugate to be used for therapeutic purposes (e.g., wherein the cyclic dinucleotide conjugate is conjugated with a therapeutic agent (e.g., a cancer therapeutic agent)).

Such embodiments are not limited to a particular definition for R1 and R2. In some embodiments, R1 and R2 are each independently a nucleotide moiety. For example, in some embodiments, R1 and R2 are each independently selected from adenine, cytosine, guanine, thymine, inosine, purine, and any derivative thereof.

Such embodiments are not limited to a particular definition for R3 and R4. In some embodiments, R3 and R4 are each independently selected from Sulfur, Nitrogen, Oxygen, and CH₂.

Such embodiments are not limited to a particular definition for R5 and R6. In some embodiments, R5 and R6 are each independently selected from NH, CH₂, Oxygen, Fluorine, Iodine, an isotope (e.g., radioisotope (eg., ₁₈F, ₁₂₃I, ₁₂₄I, ₁₂₅I, ₃₂P, ₃₅S, ₆₇Ga, etc)), a chromogenic enzyme (e.g., peroxidase, alkaline phosphatase, etc), a chromophore, a luminescent or fluorescent substance (e.g., FITC, RITC, green fluorescent protein (GFP), enhanced green fluorescent protein (EGFP), red fluorescent protein (RFP), Discosoma sp. red fluorescent protein (DsRed), cyan fluorescent protein (CFP), cyan green fluorescent protein (CGFP), yellow fluorescent protein (YFP), Cy3, Cy5, Cy7.5, etc), and a magnetic resonance imaging substance (e.g., gadolinium (Gd), super paramagentic particles or ultrasuper paramagentic particles, etc).

Such embodiments are not limited to a particular definition for R7 and R8. In some embodiments, R7 and R8 are each independently selected from

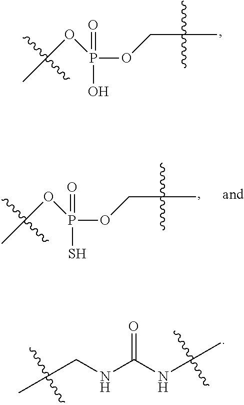 [SMILES: *COP(=O)(O)O* ; *COP(=O)(S)O* ; *CNC(=O)N*]

Such embodiments are not limited to a particular definition for R9. In some embodiments, R9 is a peptide linker or non-peptide linker that covalently links the R10 to the one of R1, R2, R5 and R6. In some embodiments, R9 is selected from

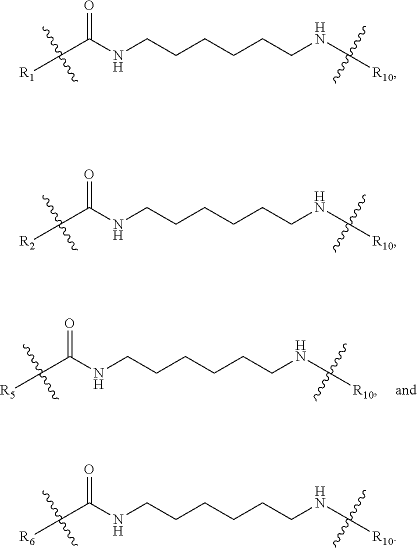

Such embodiments are not limited to a particular definition for R10. In some embodiments, R10 is selected from a therapeutic agent, a biological monitoring agent, an imaging agent, and a targeting agent. In some embodiments, R10 is Cy7. In some embodiments, R10 is Dy547.

In some embodiments, the therapeutic agent is selected from a chemotherapeutic agent, an anti-oncogenic agent, an anti-angiogenic agent, a tumor suppressor agent, an anti-microbial agent, or an expression construct comprising a nucleic acid encoding a therapeutic protein, although the present invention is not limited by the nature of the therapeutic agent. In further embodiments, the therapeutic agent is protected with a protecting group selected from photo-labile, radio-labile, and enzyme-labile protecting groups. In some embodiments, the chemotherapeutic agent is selected from a group consisting of, but not limited to, platinum complex, verapamil, podophylltoxin, carboplatin, procarbazine, mechloroethamine, cyclophosphamide, camptothecin, ifosfamide, melphalan, chlorambucil, bisulfan, nitrosurea, adriamycin, dactinomycin, daunorubicin, doxorubicin, bleomycin, plicomycin, mitomycin, bleomycin, etoposide, tamoxifen, paclitaxel, taxol, transplatinum, 5-fluorouracil, vincristin, vinblastin, bisphosphonate (e.g., CB3717), chemotherapeutic agents with high affinity for folic acid receptors, ALIMTA (Eli Lilly), and methotrexate. In some embodiments, the anti-oncogenic agent comprises an antisense nucleic acid (e.g., RNA, molecule). In certain embodiments, the antisense nucleic acid comprises a sequence complementary to an RNA of an oncogene. In preferred embodiments, the oncogene includes, but is not limited to, abl, Bcl-2, Bcl-xL, erb, fms, gsp, hst, jun, myc, neu, raf; ras, ret, src, or trk. In some embodiments, the nucleic acid encoding a therapeutic protein encodes a factor including, but not limited to, a tumor suppressor, cytokine, receptor, inducer of apoptosis, or differentiating agent. In preferred embodiments, the tumor suppressor includes, but is not limited to, BRCA1, BRCA2, C-CAM, p16, p21, p53, p73, Rb, and p27. In preferred embodiments, the cytokine includes, but is not limited to, GMCSF, IL-1, IL-2, IL-3, IL-4, IL-5, IL-6, IL-7, IL-8, IL-9, IL-10, IL-11, IL-12, IL-13, IL-14, IL-15, β-interferon, γ-interferon, and TNF. In preferred embodiments, the receptor includes, but is not limited to, CFTR, EGFR, estrogen receptor, IL-2 receptor, and VEGFR. In preferred embodiments, the inducer of apoptosis includes, but is not limited to, AdElB, Bad, Bak, Bax, Bid, Bik, Bim, Harakid, and ICE-CED3 protease. In some embodiments, the therapeutic agent comprises a short-half life radioisotope.

In some embodiments, the therapeutic agent is an anti-angiogenic agent. A variety of anti-angiogenic agents are contemplated to be useful in the compositions of the present invention including, but not limited to, Batimastat, Marimastat, AG3340, Neovastat, PEX, TIMP-1, -2, -3, -4, PAI-1, -2, uPA Ab, uPAR Ab, Amiloride , Minocycline, tetracyclines, steroids, cartilage-derived TIMP, αvβ3 Ab : LM609 and Vitaxin, RGD containing peptides, αvβ5 Ab, Endostatin, Angiostatin, aaAT, IFN-α, IFN-γ, IL-12, nitric oxide synthase inhibitors, TSP-1, TNP-470, Combretastatin A4, Thalidomide, Linomide, IFN-α, PF-4, prolactin fragment, Suramin and analogues, PPS, distamycin A analogues, FGF-2 Ab, antisense-FGF-2, Protamine, SU5416, soluble Flt-1, dominant-negative Flk-1, VEGF receptor ribosymes, VEGF Ab, Aspirin, NS-398, 6-AT, 6A5BU, 7-DX, Genistein, Lavendustin A, Ang-2, batimastat, marimastat, anti-αvβ3 monoclonal antibody (LM609) thrombospondin-1 (TSP-1) Angiostatin, endostatin, TNP-470, Combretastatin A-4, Anti-VEGF antibodies, soluble Flk-1, Flt-1 receptors, inhibitors of tyrosine kinase receptors, SU5416, heparin-binding growth factors, pentosan polysulfate, platelet-derived endothelial cell growth factor/Thymidine phosphorylase (PD-ECGF/TP), cox (e.g., cox-1 an cox-2) inhibitors (e.g., Celebrex and Vioxx), DT385 , Tissue inhibitor of metalloprotease (TIMP-1, TIMP-2), Zinc, Plasminogen activator-inhibitor-1 (PAI-1), p53 Rb, Interleukin-10 Interleukin-12, Angiopoietin-2, Angiotensin, Angiotensin II (AT2 receptor), Caveolin-1, caveolin-2, Angiopoietin-2, Angiotensin, Angiotensin II (AT2 receptor), Caveolin-1, caveolin-2, Endostatin, Interferon-alpha, Isoflavones, Platelet factor-4, Prolactin (16 Kd fragment), Thrombospondin, Troponin-1, Bay 12-9566, AG3340, CGS 27023A, CGS 27023A, COL-3, (Neovastat), BMS-275291, Penicillamine, TNP-470 (fumagillin derivative), Squalamine, Combretastatin, Endostatin, Penicillamine, Farnesyl Transferase Inhibitor (FTI), -L-778,123, -SCH_{66336,} -R115777, anti-VEGF antibody, Thalidomide, SU5416, Ribozyme, Angiozyme, SU6668, PTK787/ZK22584, Interferon-alpha, Interferon-alpha, Suramin, Vitaxin, EMD121974, Penicillamine, Tetrathiomolybdate, Captopril, serine protease inhibitors, CAI, ABT-627, CM101/ZDO101, Interleukin-12, IM862, PNU-145156E, those described in U.S. Patent App. No. 20050123605, herein incorporated by reference in its entirety, and fragments or portions of the above that retain anti-angiogenic (e.g., angiostatic or inhibitory properties).

In some embodiments, the biological monitoring agent comprises an agent that measures an effect of a therapeutic agent (e.g., directly or indirectly measures a cellular factor or reaction induced by a therapeutic agent), however, the present invention is not limited by the nature of the biological monitoring agent. In some embodiments, the monitoring agent is capable of detecting (e.g., measuring) apoptosis caused by the therapeutic agent.

In some embodiments, the imaging agent comprises a radioactive label including, but not limited to ¹⁴C, ³⁶Cl, ⁵⁷Co, ⁵⁸ Co, ⁵¹Cr, ¹²⁵I, ¹³¹I, ¹¹¹Ln, ¹⁵²Eu, ⁵⁹Fe, ⁶⁷Ga, ³²P, ¹⁸⁶Re, ³⁵S, ⁷⁵Se, Tc-99m, and ¹⁷⁵Yb. In some embodiments, the imaging agent comprises a fluorescing entity. In a preferred embodiment, the imaging agent is fluorescein isothiocyanate or 6-TAMARA. In some embodiments, the imaging agent is applicable for photoacoustic imaging and/or acoustic imaging.

In some embodiments, the targeting agent includes, but is not limited to an antibody, receptor ligand, hormone, vitamin, and antigen, however, the present invention is not limited by the nature of the targeting agent. In some embodiments, the antibody is specific for a disease-specific antigen. In some preferred embodiments, the disease-specific antigen comprises a tumor-specific antigen. In some embodiments, the receptor ligand includes, but is not limited to, a ligand for CFTR, EGFR, estrogen receptor, FGR2, folate receptor, IL-2 receptor, glycoprotein, and VEGFR. In a preferred embodiment, the receptor ligand is folic acid.

In some embodimetns, R1, R2, R3, R4, R5, R6, R7, and R8 independently combine such that the resulting chemical moiety is a cyclic dinucleotide. In some embodiments, the cyclic dinucleotide is selected from cGAMP, cdiAMP, cdiGMP, and cAIMP. Additional examples of cyclic dinucleotides are described in some detail in, e.g., U.S. Pat. Nos. 7,709,458 and 7,592,326; WO2007/054279; and Yan et al., Bioorg. Med. Chem Lett. 18: 5631 (2008)).

In some embodiments, the cyclic dinucleotide conjugate of Formula I is selected from:

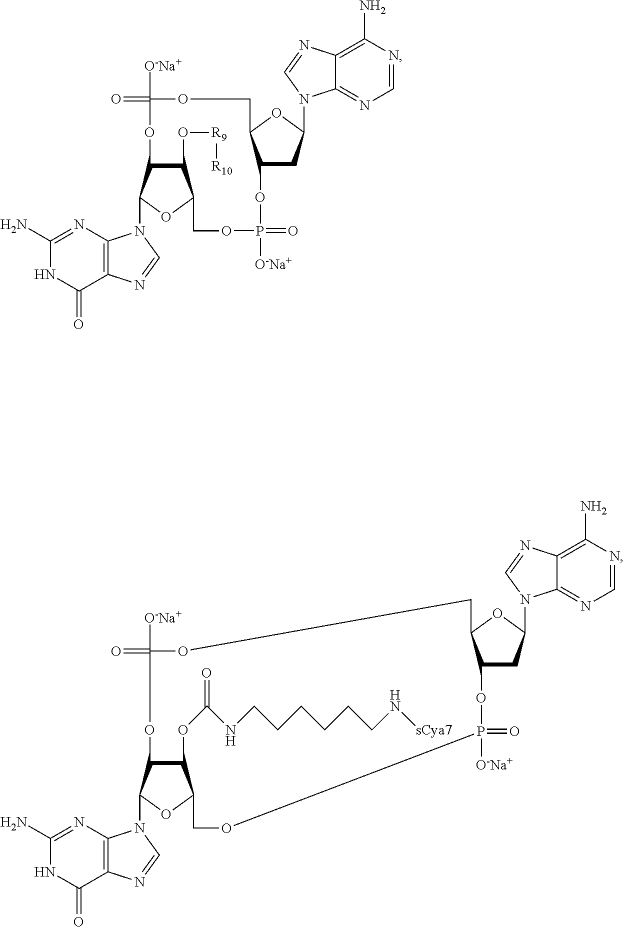

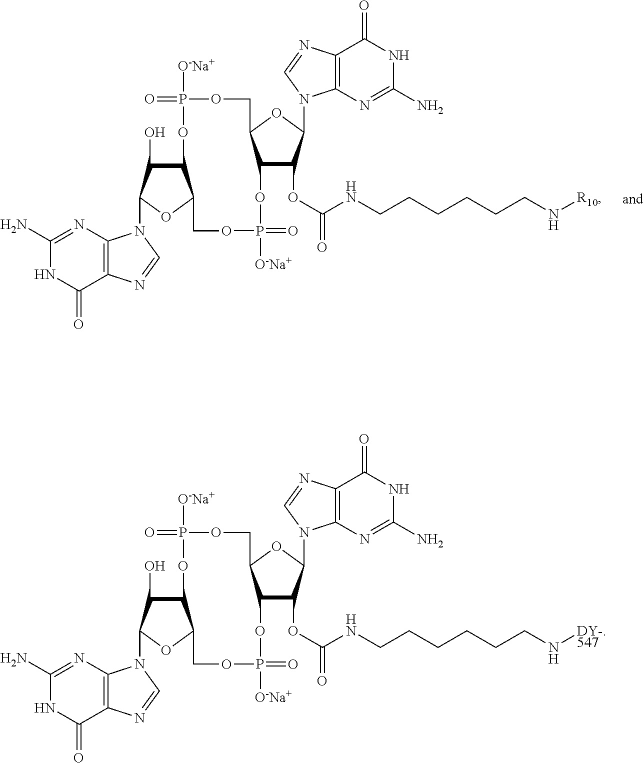

The invention further provides processes for preparing any of the cyclic dinucleotide conjugates of the present invention.

In certain embodiments, the present invention provides methods for administering a pharmaceutical composition comprising one or more cyclic dinucleotide conjugates of the present invention to a subject (e.g., a human subject) (e.g., a human subject suffering from or at risk of suffering from cancer) for purposes of imaging cancer cells (e.g., tumor cells), diagnosing a cancer, and/or treating, preventing and/or ameliorating the symptoms of a cancer.

In certain embodiments, the present invention provides a method of imaging a cancer cell comprising providing a composition comprising cyclic dinucleotides conjugated with an imaging agent as described herein, and exposing the cancer cell to the composition under conditions such that the composition interacts with and renders the cancer cell detectable. In some embodiments, the cancer cell is present in vivo. In some embodiments, the method images the cancer cell in a region beyond the primary tumor site. In some embodiments, detection of the cancer cell in a region beyond the primary tumor site is indicative of metastasis. In some embodiments, the imaging is used for staging of cancer. In some embodiments, the cyclic dinucleotides conjugated with an imaging agent are further conjugated with a therapeutic agent, biological monitoring agent, and/or a targeting agent.

In some embodimetns, the cyclic dinucleotides are conjugated with a radioisotope and are used as a contrast agent in PET imaging applications.

In some embodimetns, the cyclic dinucleotides are conjugated with a MRI imaging moiety (e.g., DOTA-Gd/Mn, NOTA-Gd/Mn, etc.), and are used as a contrast agent in MRI imaging applications. Such MRI agents are typically constructed by conjugating chelated paramagnetic ions, such as Gd(III)-diethylenetriaminepentaacetic acid (Gd(III)-DTPA), to the cyclic dinucleotide conjugates. Other paramagnetic ions that may be useful in this context of the include, but are not limited to, gadolinium, manganese, copper, chromium, iron, cobalt, erbium, nickel, europium, technetium, indium, samarium, dysprosium, ruthenium, ytterbium, yttrium, and holmium ions and combinations thereof. In some embodiments, the cylic dinucleotide conjugate is also conjugated to a targeting group, such as epidermal growth factor (EGF), to make the conjugate specifically bind to the desired cell type (e.g., in the case of EGF, EGFR-expressing tumor cells).

In some embodiments, detection of an imaging agent as determined using compositions and methods of the present invention are correlated with cancer stage and/or tumor volume. For example, in some embodiments, cyclic dinucleotides conjugated with imaging agents and/or targeting agents (as described herein) are are used to determine the stage of cancer (e.g., using a GLEASON grade or TNM staging). In other embodiments, the present invention provides targeting and identification of cancer cells (e.g., cancerous prostate cells) or tissues that permits the detection of the cancer cells and tissue in any region (e.g., in breast tissue, colon tissue, prostate, epithelium, lung, etc.) of the subject. For example, in some embodiments, the present invention detects and/or targets cancerous cells or tissue in a region outside of the primary site of cancer including, but not limited to, the vasculature and lymph nodes (e.g., the periprostatic, obturator, external iliac, hypogastric, common iliac and periaortic nodes). In some embodiments, detection of a cancerous cell outside of a primary cancer site is indicative of metastasis. Thus, in some embodiments, the present invention provides diagnostic information regarding metastasis and progression of a primary cancer.

The cyclic dinucleotide conjugates of the present invention find use in the detection and treatment of a variety of cancers. Indeed, the present invention is not limted by the type of cancer to be treated. Thus, in some embodiments, the present invention provides compositions comprising cyclic dinucleotide conjugates for the targeting and identification of angiogenesis associated with cancers (e.g., carcinomas). For example, in some embodiments, cyclic dinucleotide conjugates of the present invention further comprises a targeting agent (e.g., folic acid moiety) that associates with high affinity to a targeting agent ligand (e.g., receptor) on a cancer cell (e.g., carcinoma cells and/or solid tumor cells). In some embodiments, cyclic dinucleotides conjugated with a targeting agent (as described herein) that targets and identifies cancer cells and/or angiogenesis associated with cancer, further comprise a therapeutic agent that inhibits angiogenesis thereby treating the cancer.

In some embodiments, imaging is based on the passive or active observation of local differences in density of selected physical properties of the investigated complex matter. These differences may be due to a different shape (e.g., mass density detected by atomic force microscopy), altered composition (e.g., radiopaques detected by X-ray), distinct light emission (e.g., fluorochromes detected by spectrophotometry), different diffraction (e.g., electron-beam detected by TEM), contrasted absorption (e.g., light detected by optical methods), or special radiation emission (e.g., isotope methods), etc. Thus, quality and sensitivity of imaging depend on the property observed and on the technique used. The imaging techniques for cancerous cells have to provide sufficient levels of sensitivity to is observe small, local concentrations of selected cells. The earliest identification of cancer signatures requires high selectivity (i.e., highly specific recognition provided by appropriate targeting) and the highest possible sensitivity.

The present invention also provides a method of treating a cancer comprising administering to a subject suffering from or susceptible to cancer a therapeutically effective amount of a composition comprising cyclic dinucleotides conjugated with a therapeutic agent. In some embodiments, the cyclic dinucleotides conjugated with a therapeutic agent are further conjugated with an imaging agent, biological monitoring agent, and/or a targeting agent. In some embodiments, the composition is co-administered with an additional therapeutic agent (e.g., chemotherapeutic agent, an anti-oncogenic agent, an anti-angiogenic agent, a tumor suppressor agent, an anti-microbial agent, etc.).

Such methods are not limited by the type of cancer treated using the compositions and methods of the present invention. Indeed, a variety of cancer can be treated including, but not limited to, leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma.

The biological monitoring or sensing component of the cyclic dinucleotides conjugated with biological monitoring agents (as described herein) is one that can monitor the particular response in the tumor cell induced by an agent (e.g., a therapeutic agent provided by the therapeutic component of the cyclic dinucleotide conjugate). While the present invention is not limited to any particular monitoring system, the invention is illustrated by methods and compositions for monitoring cancer treatments. In preferred embodiments of the present invention, the agent induces apoptosis in cells and monitoring involves the detection of apoptosis. In particular embodiments, the monitoring component is an agent that fluoresces at a particular wavelength when apoptosis occurs. For example, in some embodiments, caspase activity activates green fluorescence in the monitoring component. Apoptotic cancer cells, which have turned red as a result of being targeted by a particular signature with a red label, turn orange while residual cancer cells remain red. Normal cells induced to undergo apoptosis (e.g., through collateral damage), if present, will fluoresce green.

In some embodiments, fluorescent groups such as fluorescein are employed as a biological monitoring component. Additional fluorescent dyes that find use with the present invention include, but are not limited to, acridine orange, reported as sensitive to DNA changes in apoptotic cells (Abrams et al., Development 117:29 (1993)) and cis-parinaric acid, sensitive to the lipid peroxidation that accompanies apoptosis (Hockenbery et al., Cell 75:241 (1993)). It should be noted that the fluorescent dyes are merely exemplary. It is contemplated that any agent that effectively acts as a substrate for a caspase produced as a result of apoptosis finds use with the present invention.

In some embodiments, the cyclic dinucleotide conjugates are able to specifically target a particular cell type (e.g., tumor cell). In some embodiments, the cyclic dinucleotide conjugates targets neoplastic cells through a cell surface moiety and is taken into the cell through receptor mediated endocytosis.

In some embodiments, targeting groups are conjugated to cyclic dinucleotides with either short (e.g., direct coupling), medium (e.g., using small-molecule bifunctional linkers such as SPDP, sold by Pierce Chemical Company), or long (e.g., PEG bifunctional linkers, sold by Nektar, Inc.) linkages.

Antibodies can be generated to allow for the targeting of antigens or immunogens (e.g., tumor, tissue or pathogen specific antigens) on various biological targets (e.g., pathogens, tumor cells, normal tissue). Such antibodies include, but are not limited to polyclonal, monoclonal, chimeric, single chain, Fab fragments, and an Fab expression library.

In some preferred embodiments, the antibodies recognize tumor specific epitopes (e.g., TAG-72 (Kjeldsen et al., Cancer Res. 48:2214-2220 (1988); U.S. Pat. Nos. 5,892,020; 5,892,019; and 5,512,443); human carcinoma antigen (U.S. Pat. Nos. 5,693,763; 5,545,530; and 5,808,005); TP1 and TP3 antigens from osteocarcinoma cells (U.S. Pat. No. 5,855,866); Thomsen-Friedenreich (TF) antigen from adenocarcinoma cells (U.S. Pat. No. 5,110,911); “KC-4 antigen” from human prostrate adenocarcinoma (U.S. Pat. Nos. 4,708,930 and 4,743,543); a human colorectal cancer antigen (U.S. Pat. No. 4,921,789); CA125 antigen from cystadenocarcinoma (U.S. Pat. No. 4,921,790); DF3 antigen from human breast carcinoma (U.S. Pat. Nos. 4,963,484 and 5,053,489); a human breast tumor antigen (U.S. Pat. No. 4,939,240); p97 antigen of human melanoma (U.S. Pat. No. 4,918,164); carcinoma or orosomucoid-related antigen (CORA) (U.S. Pat. No. 4,914,021); a human pulmonary carcinoma antigen that reacts with human squamous cell lung carcinoma but not with human small cell lung carcinoma (U.S. Pat. No. 4,892,935); T and Tn haptens in glycoproteins of human breast carcinoma (Springer et al., Carbohydr. Res. 178:271-292 (1988)), MSA breast carcinoma glycoprotein termed (Tjandra et al., Br. J. Surg. 75:811-817 (1988)); MFGM breast carcinoma antigen (Ishida et al., Tumor Biol. 10:12-22 (1989)); DU-PAN-2 pancreatic carcinoma antigen (Lan et al., Cancer Res. 45:305-310 (1985)); CA125 ovarian carcinoma antigen (Hanisch et al., Carbohydr. Res. 178:29-47 (1988)); YH206 lung carcinoma antigen (Hinoda et al., (1988) Cancer J. 42:653-658 (1988)).

Compositions within the scope of this invention include all pharmaceutical compositions contained in an amount that is effective to achieve its intended purpose. While individual needs vary, determination of optimal ranges of effective amounts of each component is within the skill of the art. Typically, the pharmaceutical agents (e.g., the cyclic dinucleotide conjugates described herein) may be administered to mammals, e.g. humans, orally at a dose of 0.0025 to 50 mg/kg, or an equivalent amount of the pharmaceutically acceptable salt thereof, per day of the body weight of the mammal being treated. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the inhibiting agent. The unit dose may be administered one or more times daily as one or more tablets or capsules each containing from about 0.1 to about 10 mg, conveniently about 0.25 to 50 mg of the agent (e.g., small molecule) or its solvates.

In a topical formulation, the cyclic dinucleotide conjugates described herein may be present at a concentration of about 0.01 to 100 mg per gram of carrier. In a one embodiment, such a compound is present at a concentration of about 0.07-1.0 mg/ml, for example, about 0.1-0.5 mg/ml, and in one embodiment, about 0.4 mg/ml.

In addition to administering the cyclic dinucleotide conjugates described herein as a raw chemical, it may be administered as part of a pharmaceutical preparation containing suitable pharmaceutically acceptable carriers comprising excipients and auxiliaries which facilitate processing of the compound into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active mimetic peptide(s), together with the excipient.

The pharmaceutical compositions of the invention may be administered to any patient that may experience the beneficial effects of one or more of compounds of the present invention. Foremost among such patients are mammals, e.g., humans, although the invention is not intended to be so limited. Other patients include veterinary animals (cows, sheep, pigs, horses, dogs, cats and the like).

The pharmaceutical compositions comprising the cyclic dinucleotide conjugates described herein may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner that is itself known, for example, by means of conventional mixing, granulating, dragee-making, dissolving, or lyophilizing processes. Thus, pharmaceutical preparations for oral use can be obtained by combining the active mimetic peptides with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye-stuffs or pigments may be added to the tablets or dragee coatings, for example, for identification or in order to characterize combinations of active mimetic peptide doses.

Other pharmaceutical preparations that can be used orally include push-fit capsules made of gelatin, as well as soft, sealed capsules made of gelatin and a plasticizer such as glycerol or sorbitol. The push-fit capsules can contain the active mimetic peptides in the form of granules that may be mixed with fillers such as lactose, binders such as starches, and/or lubricants such as talc or magnesium stearate and, optionally, stabilizers. In soft capsules, the active mimetic peptides are in one embodiment dissolved or suspended in suitable liquids, such as fatty oils, or liquid paraffin. In addition, stabilizers may be added.

Possible pharmaceutical preparations that can be used rectally include, for example, suppositories, which consist of a combination of one or more of the active mimetic peptides with a suppository base. Suitable suppository bases are, for example, natural or synthetic triglycerides, or paraffin hydrocarbons. In addition, it is also possible to use gelatin rectal capsules that consist of a combination of the active mimetic peptides with a base. Possible base materials include, for example, liquid triglycerides, polyethylene glycols, or paraffin hydrocarbons.

Suitable formulations for parenteral administration include aqueous solutions of the active mimetic peptides in water-soluble form, for example, water-soluble salts and alkaline solutions. In addition, suspensions of the active mimetic peptides as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions of this invention are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Ointments may be formulated by mixing a solution of the active ingredient in a vegetable oil such as almond oil with warm soft paraffin and allowing the mixture to cool. A typical example of such an ointment is one that includes about 30% almond oil and about 70% white soft paraffin by weight. Lotions may be conveniently prepared by dissolving the active ingredient, in a suitable high molecular weight alcohol such as propylene glycol or polyethylene glycol.

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXPERIMENTAL

### Example I

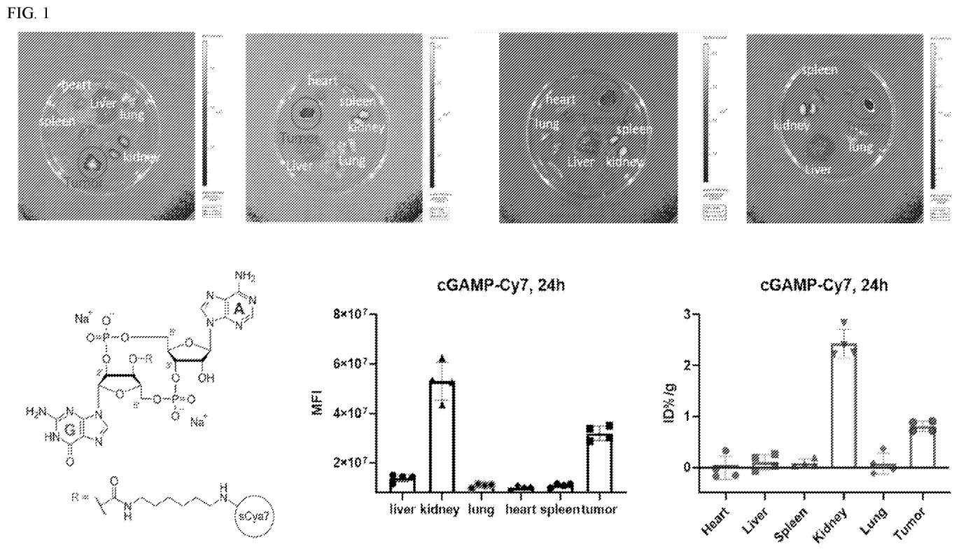

Mice bearing CT26 colon carcinoma were administered via intravenous route with cGAMP-Cy7 (FIG. 1). After 24 hr, mice were euthanized, and the major organs were visualized for cGAMP-Cy7 signal using IVIS. Strong cGAMP-Cy7 signal was observed in tumors and kidneys (FIG. 1).

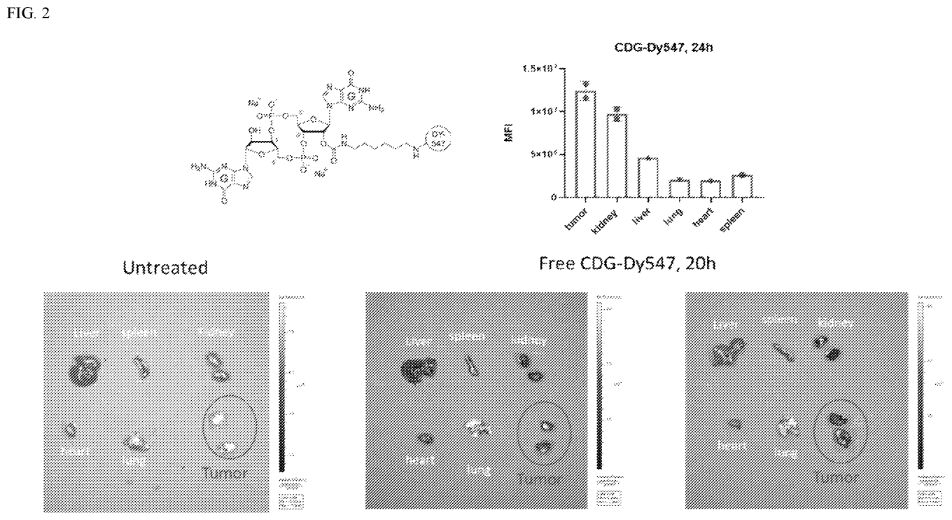

Mice bearing CT26 colon carcinoma were administered via intravenous route with CDG-Cy547 (FIG. 2). After 24 hr, mice were euthanized, and the major organs were visualized for CDG-Cy547 signal using IVIS. Strong CDG-Cy547 signal was observed in tumors and kidneys (FIG. 2).

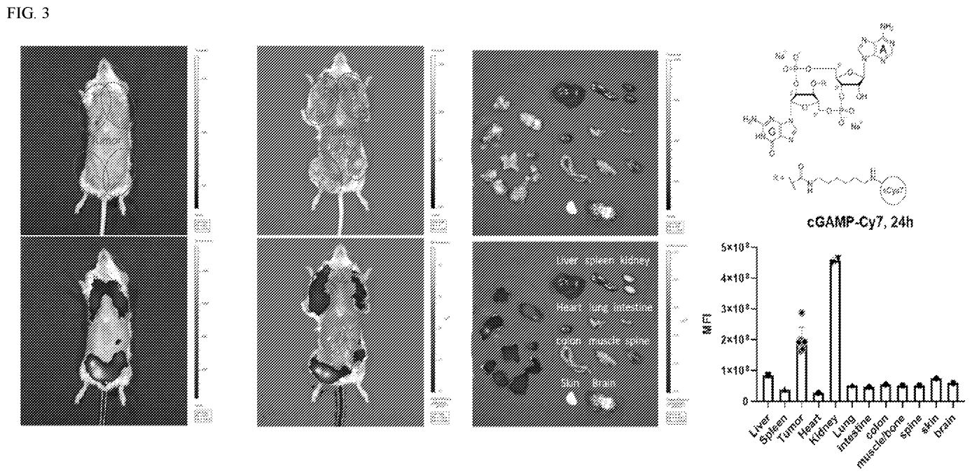

Mice bearing MMTV-PyMT spontaneous breast cancer were administered via intravenous route with cGAMP-Cy7 (FIG. 3). After 24 hr, mice were euthanized, and the major organs were visualized for cGAMP-Cy7 signal using IVIS. Strong cGAMP-Cy7 signal was observed in tumors and kidneys (FIG. 3).

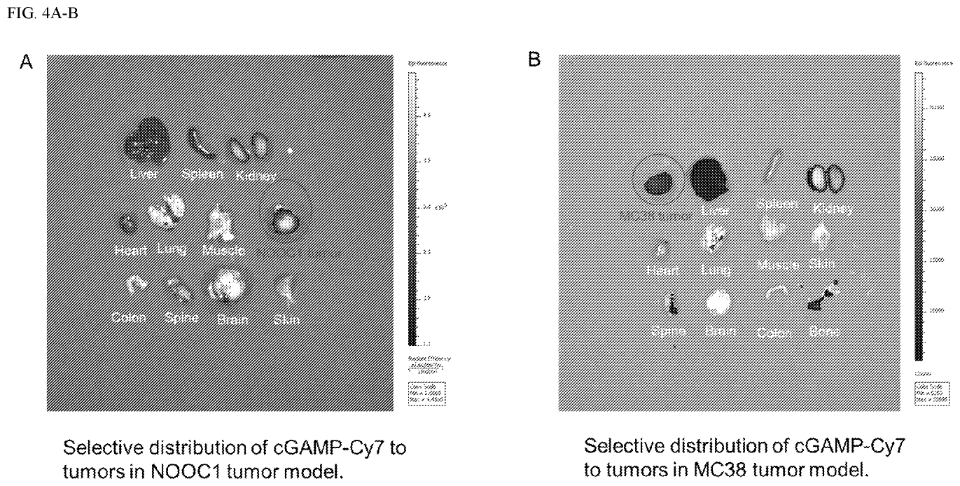

Mice bearing NOOC1 subcutaneous head and neck carcinoma were administered via intravenous route with cGAMP-Cy7 (FIG. 4A). After 24 hr, mice were euthanized, and the major organs were for visualized for cGAMP-Cy7 signal using IVIS. Strong cGAMP-Cy7 signal was observed in tumors and kidneys (FIG. 4A).

Mice bearing MC38 colon carcinoma were administered via intravenous route with cGAMP-Cy7 (FIG. 4B). After 24 hr, mice were euthanized and the major organs werefor visualized for cGAMP-Cy7 signal using IVIS. Strong cGAMP-Cy7 signal was observed in tumors and kidneys (FIG. 4B).

### Material and Method:

All animals were cared for following federal, state, and local guidelines. All work performed on animals was in accordance with and approved by the Institutional Animal Care & Use Committee (IACUC) at the University of Michigan, Ann Arbor. For CT26 murine tumor model, female BALB/c mice of age 6-8 weeks (Jackson Laboratories) were inoculated with CT26 colon cancer cells subcutaneously. For MMTV-PyMT model, spontaneous tumors formed around mammary pads spontaneously over time. For NOOC1 and MC38 murine tumor model, female C57BL mice of age 6-8 weeks (Jackson Laboratories) were inoculated with NOOC1 and MC38 cancer cells subcutaneously. Model CDN conjugates, cGAMP-Cy7 (BioLog) and CDG-Dy547 (BioLog), were dissolved in 10% sucrose. When tumor sizes were >100 mm³, 4 nmol cGAMP-Cy7 and CDG-Dy547 in 150 ul 10% sucrose solution were administered intravenously (IV) via tail vein. After 24 h of injection, mice were euthanized and visualized by IVIS fluorescence imaging. Tissue distribution of cGAMP-Cy7 and CDG-Dy547 was quantified by measuring the mean fluorescence intensity of the major organs.

Having now fully described the invention, it will be understood by those of skill in the art that the same can be performed within a wide and equivalent range of conditions, formulations, and other parameters without affecting the scope of the invention or any embodiment thereof. All patents, patent applications and publications cited herein are fully incorporated by reference herein in their entirety.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### INCORPORATION BY REFERENCE

The entire disclosure of each of the patent documents and scientific articles referred to herein is incorporated by reference for all purposes.

What is claimed is:

1. A composition comprising a cyclic dinucleotide conjugate encompassed within Formula I: 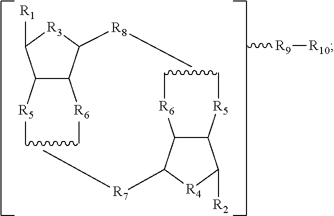
including pharmaceutically acceptable salts, solvates, and/or prodrugs thereof,
wherein each of R1, R2, R3, R4, R5, R6, R7, R8, R9 and R10 independently include any chemical moiety that permits the resulting compound to effectively function within cancer imaging, cancer diagnosis and/or cancer-targeted delivery of therapeutic agents.

2. The composition of claim 1, wherein each of R1, R2, R3, R4, R5, R6, R7, R8, R9 and R10 independently include any chemical moiety that permits the resulting cyclic dinucleotide conjugate to be used as a contrast agent for PET imaging of cancer.

3. The composition of claim 1, wherein each of R1, R2, R3, R4, R5, R6, R7, R8, R9 and R10 independently include any chemical moiety that permits the resulting cyclic dinucleotide conjugate to be used as contrast agents of MR imaging of cancer.

4. The composition of claim 1, wherein each of R1, R2, R3, R4, R5, R6, R7, R8, R9 and R10 independently include any chemical moiety that permits the resulting cyclic dinucleotide conjugate to be used for imaging purposes (e.g., PET imaging, MR imaging, fluorescent imaging, photoacoustic imaging, acoustic imaging, etc).

5. The composition of claim 1, wherein each of R1, R2, R3, R4, R5, R6, R7, R8, R9 and R10 independently include any chemical moiety that permits the resulting cyclic dinucleotide conjugate to be used for diagnostic purposes (e.g., histochemical staining of tumor, intraoperative imaging of tumor to aid surgery therapy, predicting prognosis, etc.).

6. The composition of claim 1, wherein each of R1, R2, R3, R4, R5, R6, R7, R8, R9 and R10 independently include any chemical moiety that permits the resulting cyclic dinucleotide conjugate to be used for therapeutic purposes (e.g., wherein the cyclic dinucleotide conjugate is conjugated with a therapeutic agent (e.g., a cancer therapeutic agent)).

7. The composition of claim 1, wherein R1 and R2 are each independently a nucleotide moiety.

8. The composition of claim 1, wherein R1 and R2 are each independently selected from adenine, cytosine, guanine, thymine, inosine, purine, and any derivative thereof

9. The composition of claim 1, wherein R3 and R4 are each independently selected from Sulfur, Nitrogen, Oxygen, and CH₂.

10. The composition of claim 1, wherein R5 and R6 are each independently selected from NH, CH₂, Oxygen, Fluorine, Iodine, an isotope (e.g., radioisotope (eg., ₁₈F, ₁₂₃I, ₁₂₄I, ₁₂₅I, ₃₂P, ₃₅S, ₆₇Ga, etc)), a chromogenic enzyme (e.g., peroxidase, alkaline phosphatase, etc), a chromophore, a luminescent or fluorescent substance (e.g., FITC, RITC, green fluorescent protein (GFP), enhanced green fluorescent protein (EGFP), red fluorescent protein (RFP), Discosoma sp. red fluorescent protein (DsRed), cyan fluorescent protein (CFP), cyan green fluorescent protein (CGFP), yellow fluorescent protein (YFP), Cy3, Cy5, Cy7.5, etc), and a magnetic resonance imaging substance (e.g., gadolinium (Gd), super paramagentic particles or ultrasuper paramagentic particles, etc).

11. The composition of claim 1, wherein R7 and R8 are each independently selected from

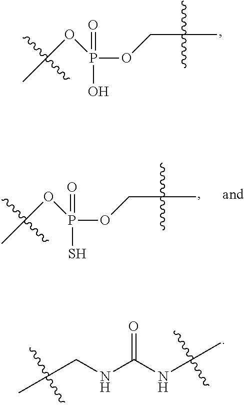 [SMILES: *COP(=O)(O)O* ; *COP(=O)(S)O* ; *CNC(=O)N*]

12. The composition of claim 1, wherein R9 is a peptide linker or non-peptide linker that covalently links the R10 to the one of R1, R2, R5 and R6,

13. The composition of claim 12, wherein R9 is selected from

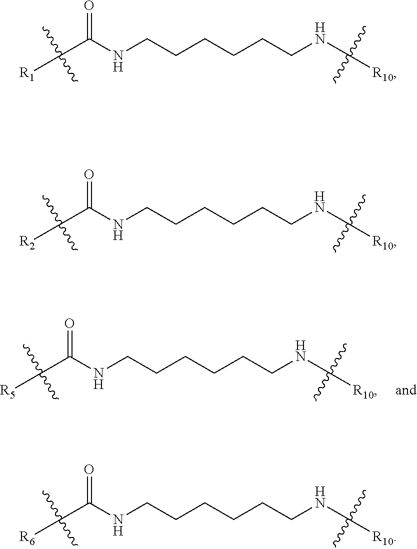

14. The composition of claim 1, wherein R10 is Cy7 or Dy547.

15. The composition of claim 1, wherein R10 is selected from a therapeutic agent, a biological monitoring agent, an imaging agent, and a targeting agent.

16. The composition of claim 15, wherein the therapeutic agent is selected from a chemotherapeutic agent, an anti-oncogenic agent, an anti-angiogenic agent, and a tumor suppressor agent.

17. The composition of claim 16, wherein the chemotherapeutic agent is selected from a group consisting of a platinum complex, verapamil, podophylltoxin, carboplatin, procarbazine, mechloroethamine, cyclophosphamide, camptothecin, ifosfamide, melphalan, chlorambucil, bisulfan, nitrosurea, adriamycin, dactinomycin, daunorubicin, doxorubicin, bleomycin, plicomycin, mitomycin, bleomycin, etoposide, tamoxifen, paclitaxel, taxol, transplatinum, 5-fluorouracil, vincristin, vinblastin, bisphosphonate (e.g., CB3717), chemotherapeutic agents with high affinity for folic acid receptors, ALIMTA (Eli Lilly), and methotrexate.

18. The composition of claim 15, wherein the imaging agent is selected from a radioactive label (e.g., ¹⁴C, ³⁶Cl, ⁵⁸ Co, ⁵⁷Co, ⁵¹Cr, ¹²⁵I, ¹³¹I, ¹¹¹Ln, ¹⁵²Eu, ⁵⁹Fe, ⁶⁷Ga, ³²P, ¹⁸⁶Re, ³⁵S, ⁷⁵Se, Tc-99m, and ¹⁷⁵Yb).

19. The composition of claim 15, wherein the imaging agent is a fluorescing entity (e.g., fluorescein isothiocyanate or 6-TAMARA).

20. The composition of claim 15, wherein the imaging agent is applicable for photoacoustic imaging and/or acoustic imaging.

21. The composition of claim 15, wherein the targeting agent is selected from an antibody, a receptor ligand (e.g., a ligand for CFTR, EGFR, estrogen receptor, FGR2, folate receptor, IL-2 receptor, glycoprotein, and VEGFR, a hormone, a vitamin, and an antigen (e.g., tumor specific antigen).

22. The composition of claim 1, wherein R1, R2, R3, R4, R5, R6, R7, and R8 independently combine such that the resulting chemical moiety is a cyclic dinucleotide.

23. The composition of claim 22, wherein the cyclic dinucleotide is selected from cGAMP, cdiAMP, cdiGMP, and cAIMP, or variations thereof.

24. The composition of claim 1, wherein the cyclic dinucleotide conjugate is selected from:

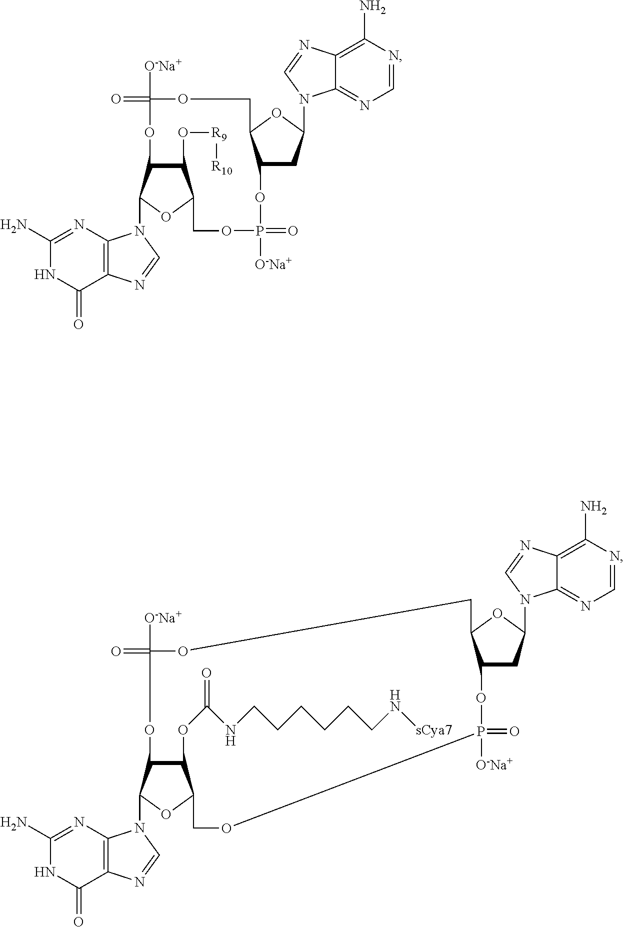

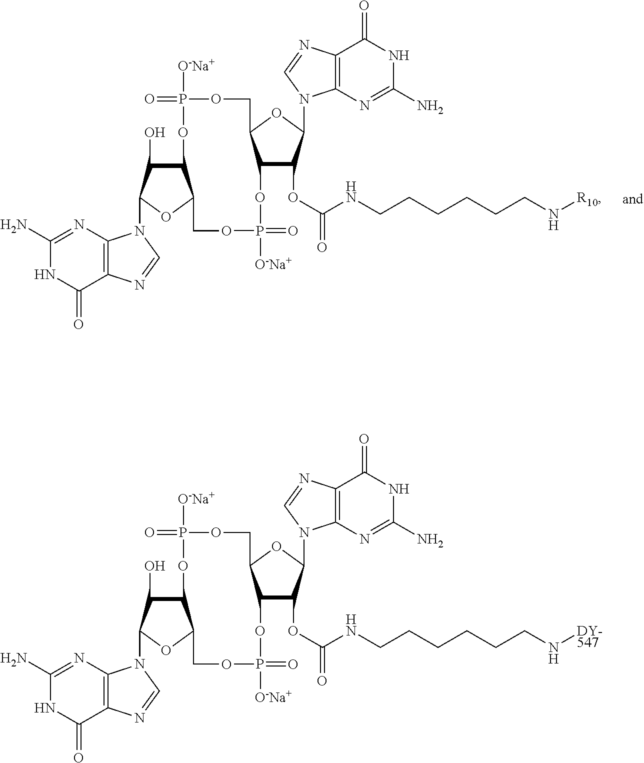

25. A method for imaging a cancer cell comprising providing a composition of claim 1 wherein R10 is an imaging agent, and exposing the cancer cell to the composition under conditions such that the composition interacts with and renders the cancer cell detectable.

26. The method of claim 25, wherein the cancer cell is within a tumor.

27. The method of claim 25, wherein the cyclic dinucleotide conjugate is further conjugated with a therapeutic agent, a biological monitoring agent, and/or a targeting agent.

28. The method of claim 25, wherein the cyclic dinucleotides are conjugated with a radioisotope and the imaging is PET imaging.

29. The method of claim 25, wherein the cyclic dinucleotides are conjugated with a MRI imaging moiety (e.g., DOTA-Gd/Mn, NOTA-Gd/Mn, etc.), and the imaging is MR1 imaging.

30. A method for treating cancer in a subject, comprising administering to a subject suffering from or susceptible to cancer a therapeutically effective amount of a composition of claim 1 wherein R10 is a therapeutic agent.

31. The method of claim 30, wherein the cyclic dinucleotide conjugate is further conjugated with an imaging agent, a biological monitoring agent, and/or a targeting agent.

32. The method of claim 30, wherein the composition is co-administered with an additional therapeutic agent (e.g., chemotherapeutic agent, an anti-oncogenic agent, an anti-angiogenic agent, a tumor suppressor agent, an anti-microbial agent, etc.).

33. The method of claim 30, wherein the cancer is one or more of leukemia, acute leukemia, acute lymphocytic leukemia, acute myelocytic leukemia, myeloblastic, promyelocytic, myelomonocytic, monocytic, erythroleukemia, chronic leukemia, chronic myelocytic, (granulocytic) leukemia, chronic lymphocytic leukemia, Polycythemia vera, lymphoma, Hodgkin's disease, non-Hodgkin's disease, Multiple myeloma, Waldenstrom's macroglobulinemia, Heavy chain disease, solid tumors, sarcomas and carcinomas, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, and neuroblastomaretinoblastoma.

34. A kit comprising a composition as described in claim 1, and one or more of (1) a container, pack, or dispenser, (2) one or more additional agents selected from a chemotherapeutic agent, an anti-oncogenic agent, an anti-angiogenic agent, a tumor suppressor agent, an anti-microbial agent, etc., and (3) instructions for administration.
